(19) United States
(12) Patent Application Publication
(10) Pub. No.: US 2022/0000834 A1
(43) Pub. Date: Jan. 6, 2022

(54) NOVEL ADMINISTRATION METHOD

(71) Applicant: SUMITOMO DAINIPPON PHARMA CO., LTD., Osaka-shi, Osaka (JP)

(72) Inventors: Kiyokazu IWATA, Kobe (JP); Takashi NAKAGAWA, Suita (JP); Shoji OGAWA, Suita (JP); Mitsuhiro INOUE, Kobe (JP)

(73) Assignee: SUMITOMO DAINIPPON PHARMA CO., LTD., Osaka-shi, Osaka (JP)

(21) Appl. No.: 17/293,268

(22) PCT Filed: Dec. 11, 2019
(86) PCT No.: PCT/JP2019/048408

§ 371 Date: May 12, 2021

(51) Int. Cl. A61K 31/352; A61K 9/70; A61K 9/00; A61P 25/28

(52) U.S. Cl. CPC A61K 31/352; A61P 25/28; A61K 9/0024; A61K 9/7007

(57) ABSTRACT

The present invention addresses the problem of providing a formulation for applying a semaphorin inhibitor without surgery to remove the dura mater. A sheet formulation comprising a semaphorin inhibitor for treating spinal cord injury or brain injury by epidural administration is provided.

### TECHNICAL FIELD

The present invention relates to a novel sheet preparation and a novel administration method using the novel sheet preparation in a semaphorin inhibitor. The present invention relates to a sheet preparation suitable for a novel method for administering a semaphorin inhibitor.

### BACKGROUND ART

Around 20 types of semaphorin have been found out until now, and a gene cluster belonging to a subfamily called class 3 has a function of suppressing the growth of neurite growth cone, and the like and has been particularly studied. It is known that the class 3 semaphorin (Sema3A) induces regression of nerve cells at a concentration as low as 10 pM. A low molecular weight compound which inhibits Sema3A is known as a semaphorin inhibitor. For example, compound A represented by formula (1), which is one of the semaphorin inhibitors, accelerates the regeneration of nerves in an injured part. As an example of applications, improvement in spinal cord injury is presumed (Patent Document 1 and Patent Document 2).

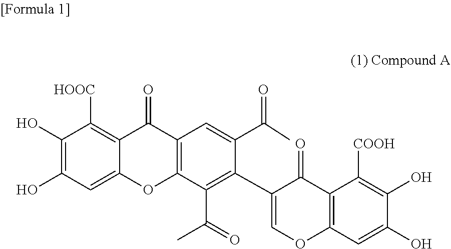 [SMILES: CC(=O)c1cc2c(=O)c3c(C(=O)O)c(O)c(O)cc3oc2c(C(C)=O)c1-c1coc2cc(O)c(O)c(C(=O)O)c2c1=O]

Meanwhile, it is known that a method for administering a semaphorin inhibitor is preferably a method for administering a semaphorin inhibitor locally to a lesion site. Therefore, when a semaphorin inhibitor is used as a therapeutic agent for spinal cord injury, a surgical operation for removing the dura mater is necessary.

The dura mater is the outermost membrane among three layers of meninges covering the brain or the spinal cord. The dura mater is a tough membrane containing a large amount of collagenous fibrils, and it is generally difficult to administer a medicine by passing through the dura mater.

### PRIOR ART DOCUMENTS

### Patent Document

Patent Document 1: International Publication No. WO2002/009756 pamphlet
Patent Documents 2: International Publication No. WO2012/018069 pamphlet

### Patent Documents 3: JP 62-174007 A

### SUMMARY OF INVENTION

### Technical Problem

As mentioned above, when a semaphorin inhibitor is used as a therapeutic agent for spinal cord injury, a surgical operation for removing the dura mater is necessary, but the surgical operation for removing the dura mater imposes a heavy physical burden on a subject. Meanwhile, means for applying a semaphorin inhibitor without performing a surgical operation for removing the dura mater has not existed until now.

An object of the present invention is to provide a preparation for applying a semaphorin inhibitor without performing a surgical operation for removing the dura mater in view of the above-mentioned current situation.

### Solution to Problem

The present inventors have extensively and intensively studied in view of the above-mentioned object and consequently found that a semaphorin inhibitor can be applied without surgically removing the dura mater by using a certain type of preparation. The present inventors have further investigated and consequently completed the present invention.

That is, the present invention relates to at least the following inventions:

[1] A sheet preparation for treating spinal cord injury or brain injury by epidural administration, comprising a semaphorin inhibitor.
[2] The sheet preparation of the above-mentioned [1], comprising silicone as a substrate.
[3] The sheet preparation of the above-mentioned [1] or [2], further comprising a water-soluble additive.
[4] The sheet preparation of the above-mentioned [3], wherein the water-soluble additive comprises one or more amino acids.
[5] The sheet preparation of the above-mentioned [4], wherein the amino acid is alanine or leucine.
[6] The sheet preparation of any one of the above-mentioned [1] to [5], wherein the semaphorin inhibitor is a semaphorin 3A inhibitor.
[7] The sheet preparation of any one of the above-mentioned [1] to [5], wherein the semaphorin inhibitor is compound A represented by formula (1):

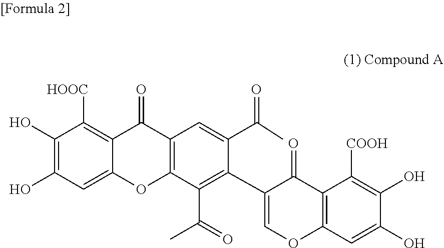 [SMILES: CC(=O)c1cc2c(=O)c3c(C(=O)O)c(O)c(O)cc3oc2c(C(C)=O)c1-c1coc2cc(O)c(O)c(C(=O)O)c2c1=O]

[8] A sheet preparation, comprising compound A represented by formula (1):

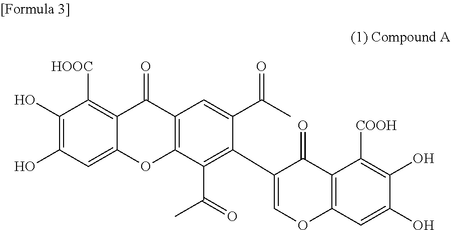 [SMILES: CC(=O)c1cc2c(=O)c3c(C(=O)O)c(O)c(O)cc3oc2c(C(C)=O)c1-c1coc2cc(O)c(O)c(C(=O)O)c2c1=O]

[9] The sheet preparation according to [8], comprising silicone as a substrate.
[10] The sheet preparation according to [8] or [9], further comprising a water-soluble additive.
[11] The sheet preparation according to [10], comprising one or more amino acids as the water-soluble additive.
[12] The sheet preparation according to [11], wherein the one or more amino acids are alanine or leucine.
[13] A method for treating spinal cord injury or brain injury, wherein a therapeutically effective amount of the sheet preparation according to any one of the above-mentioned [8] to [12] is epidurally administered to a patient in need of treatment.
[14] Use of the sheet preparation according to any one of the above-mentioned [8] to [12] for producing a therapeutic agent for spinal cord injury or brain injury to be epidurally administered.
[15] A therapeutic agent for spinal cord injury or brain injury, comprising the sheet preparation according to any one of the above-mentioned [8] to [12], wherein the therapeutic agent is epidurally administered.

### Advantageous Effects of Invention

According to a sheet preparation of the present invention, the application to an affected part of spinal cord injury or brain injury and/or its vicinity enables a semaphorin inhibitor to reach the affected part without removing the dura mater surgically. According to the present invention, the effect of greatly reducing a physical burden of a patient when the semaphorin inhibitor is administered is therefore produced.

According to a sheet preparation containing silicone as a substrate among sheet preparations of the present invention, the semaphorin inhibitor can be delivered to the affected part more efficiently.

According to a sheet preparation containing a water-soluble additive, especially one or more amino acids, among the sheet preparations of the present invention, the semaphorin inhibitor can be delivered to the affected part still more efficiently. A sheet preparation of the present invention wherein the above-mentioned one or more amino acids are alanine or leucine is particularly excellent in the efficiency of the delivery of the semaphorin inhibitor to the affected part.

A water-soluble compound such as the semaphorin inhibitor which is an active ingredient of the present invention is hardly dissolved in a hydrophobic polymer carrier, and cannot be diffused or released autonomously. Therefore, a completely different release mechanism from that of a fat-soluble drug is generally necessary.

Examples of a general technique for releasing a water-soluble drug from a hydrophobic polymer carrier include a technique for releasing a water-soluble drug from pores in a reservoir type preparation. Besides, there is also a type in which a drug is dispersed in a carrier. For this type of preparation, a phenomenon in which drug particles which exist on the surface are first dissolved in water in surrounding tissue, and drug particles in contact with these are subsequently dissolved is repeated to form continuous water channels, and the drug particles are diffused in the channels to release the drug. Also, since cracking occurs due to a difference in osmotic pressure made in a preparation at this time, channel formation is promoted, and release is further promoted by a pushing-out effect of swelling. For this reason, this type is characterized in that particles in the carrier need to be close, the difference in osmotic pressure needs to be made in the preparation, and the water-soluble drug or a water-soluble additive needs to be incorporated in a certain amount or more to maintain the release. As such an example, a method for controlling drug release from silicone by adding albumin has been reported (Patent Document 3).

However, the release control of such a water-soluble drug by a release mechanism is very difficult, and in general, water-soluble drugs are released with a first order release profile in which the drug is initially released with a significantly high release rate to cause burst release, and the release amount then decreases over time. Therefore, the steady sustained release for a long period of time is difficult.

Meanwhile, according to a sheet preparation containing silicone as a substrate and further containing a water-soluble additive, especially one or more amino acids, among the sheet preparations of the present invention, a semaphorin inhibitor can be delivered to an affected part still more efficiently. The above-mentioned sheet preparation of the present invention wherein the one or more amino acids are alanine or leucine produces the effect of delivering the semaphorin inhibitor to the affected part at a particularly good efficiency.

Since pliability is secured in the above-mentioned sheet preparation of the present invention further containing the one or more amino acids, the sheet preparation is excellent in conformability to the affected part or its vicinity, and the effect of also enhancing the degree of adhesion to the affected part or its vicinity is therefore also produced. Such an effect is produced still more remarkably in the above-mentioned sheet preparation of the present invention wherein the one or more amino acids are alanine or leucine.

### BRIEF DESCRIPTION OF DRAWINGS

FIG. 1 shows the results of a drug release test using Preparation Example 1 (Test Preparation 1-1, which is an example of a sheet preparation of the present invention)(Test Example 1). The vertical axis of the graph represents the cumulative release rate of compound A, and the horizontal axis represents elapsed time after start of the test.

FIG. 2 shows the results of a drug release test using Preparation Example 2 (Test Preparation 2-1, which is an example of a sheet preparation of the present invention)(Test Example 2). The vertical axis of the graph represents the cumulative release rate of compound A, and the horizontal axis represents elapsed time after start of the test.

FIG. 3 shows the results of BBB score evaluation using a rat spinal cord injury model with Preparation Example 2 (Test Preparation 2-2, which is an example of a sheet preparation of the present invention) and Preparation Example 3 (Comparative Example: Test Preparation 3-2) (Example 1). The vertical axis of the graph represents the cumulative release rate of compound A, and the horizontal axis represents elapsed time after start of the test.

FIG. 4 shows the results of an evaluation test on the transfer of a drug to the spinal cord by indwelling the drug outside the rat dura mater spinalis with Preparation Example 2 (Test Preparation 2-3, which is an example of a sheet preparation of the present invention) (Example 2).

FIG. 5 shows the results of canine and swine cerebral dura mater permeability evaluation tests using a franz cell with Preparation Example 1 (Test Preparation 1-2, which is an example of a sheet preparation of the present invention) (Example 3).

### DESCRIPTION OF EMBODIMENTS

The present invention relates to a sheet preparation for treating spinal cord injury or brain injury by epidural administration, comprising a semaphorin inhibitor.

Close adhesion of the sheet preparation containing the semaphorin inhibitor to the outside of the dura mater enables the increase in the concentration of the semaphorin inhibitor, especially compound A, in the spinal cord to a level at which a neurite elongation inhibiting effect of semaphorin 3A can be fully suppressed in an in vitro test using cells. The present inventors have confirmed a pharmacological effect in an in vivo test using mice.

The present inventors have found that the use of the sheet preparation of the present invention enables the achievement of a pharmacological effect equivalent to that of a preparation intradurally administered.

Further, the present inventors have also confirmed that when the sheet preparation of the present invention was used, the semaphorin inhibitor hardly transfers to blood, and thus the semaphorin inhibitor was locally administered. Furthermore, the present inventors have found a sheet preparation which enables a long-term and efficient release of the semaphorin inhibitor.

The present invention is based on these new findings.

The use of a sheet preparation has not been even attempted until now as a dosage form for applying the semaphorin inhibitor to spinal cord injury or brain injury.

—Configuration of Sheet Preparation of the Present Invention

The sheet preparation of the present invention contains the semaphorin inhibitor as an active ingredient and optionally has a configuration in which pharmaceutically acceptable ingredients other than the semaphorin inhibitor are carried on the substrate with the semaphorin inhibitor. As long as the sheet preparation can be provided for treatment of spinal cord injury or brain injury by epidural administration, its configuration is not particularly limited.

The semaphorin inhibitor is not particularly limited, and the examples thereof include various compounds described in JP 2016-037472 A and compound A. As the semaphorin inhibitor in the sheet preparation of the present invention, compound A is preferable. Compound A has the following structure:

### [Formula 4]

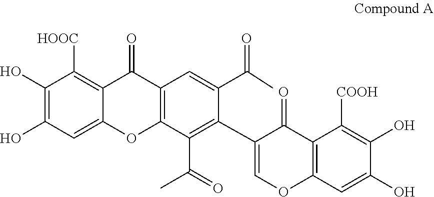 [SMILES: CC(=O)c1cc2c(=O)c3c(C(=O)O)c(O)c(O)cc3oc2c(C(C)=O)c1-c1coc2cc(O)c(O)c(C(=O)O)c2c1=O]

Compound A can be obtained by the culture of Penicillium sp. strain SPF-3059, total chemical synthesis, or chemical conversion by a well-known synthesizing method using a substance obtained by the culture or the total synthesis for a raw material.

As the culture, compound A can be obtained efficiently by culturing a fungus strain SPF-3059 belonging to Penicillium separated from soil in Osaka-fu [the present fungus strain has been deposited with the International Patent Organism Depositary (chuo 6, 1-1-1, Higashi, Tsukuba-shi, Ibaraki-ken, 305-8566), the independent administrative institution National Institute of Advanced Industrial Science and Technology, the Ministry of Economy, Trade and Industry under the accession number FERM BP-7663 on Jul. 13, 2001 based on Budapest Treaty on the International Recognition of the Deposit of Microorganisms for the Purposes of Patent Procedure]. The compound can be specifically obtained in accordance with a method described in International Publication No. WO02/09756 pamphlet (Patent Document 1) or International Publication No. WO03/062243 pamphlet.

As the total synthesis, compound A can be obtained in accordance with a method described in JP 2008-13530 A.

Although the substrate in the sheet preparation of the present invention is not limited, a biocompatible hydrophobic polymer is illustrated as an ingredient of the substrate. Although hydrophobic polymers are roughly classified into non-biodegradable hydrophobic polymers and biodegradable hydrophobic polymers, any hydrophobic polymer may be used in the sheet preparation of the present invention. Although examples are shown below, the hydrophobic polymers are not limited to these. That is, examples of the non-biodegradable polymers include silicone and polyurethane, and examples of biodegradable polymers include polylactic acid, polyglycolic acid, polycaprolactone, and a copolymer thereof.

Silicone is preferable among these hydrophobic polymers. The sheet preparation of the present invention containing silicone as the ingredient of the substrate is preferable since such a sheet preparation enables the semaphorin inhibitor to deliver to the affected part in a still more efficient manner and/or a sustained-release manner. Not theoretically bound, it is supposed that it is partly because the use of a hydrophobic polymer such as silicone as the substrate makes a difference in osmotic pressure efficiently in the sheet preparation and enhances permeability of a xanthone compound such as compound A in the dura mater.

When silicone is used as the substrate, the semaphorin inhibitor and an additive are dispersed and contained in silicone.

Silicone is a material which has been used as a raw material of artificial organs or medical tools for a long time as a raw material excellent in biocompatibility, and is also excellent in safety. Silicone includes silicone in various forms such as a form of oil, a form of gel, and a form of rubber depending on differences in the degree of polymerization of siloxane bonds and the substituent. Silicone used for the sheet preparation of the present invention is not limited, and may be silicone solidified by hardening silicone in the form of liquid, the form of oil, and the form of gel. As the silicone, liquid silicone is preferable. As the liquid silicone, for example, a SILASTIC Q7-4750A component and a SILASTIC Q7-4750B component of polydimethylsiloxane manufactured by Dow Corning, and MED-4750 manufactured by Nusil can be used. As silicone used for the sheet preparation of the present invention, the Q7-4750A component and the Q7-4750B component are preferable, and silicone using these in combination is more preferable.

The blending proportion of the substrate such as silicone in the sheet preparation of the present invention is not limited, and is, for example, 30% to 90%, preferably 35% to 75%, and more preferably 40% to 60%. The blending proportions of ingredients herein are the weight ratios of ingredients to the weight of the whole body of the sheet preparation containing the active ingredient in terms of % unless otherwise specified.

Other Ingredients

The sheet preparation of the present invention may contain other pharmaceutically acceptable ingredients. The other pharmaceutically acceptable ingredients are additives which are not particularly limited. However, examples of the additives include ordinary pharmaceutically acceptable carrier, and an excipient, a diluent, a pH buffer, a tonicity adjusting agent, a binder, a fluidizer, a lubricant, a solubilizer, a dissolving aid, a thickener, a dispersing agent, a stabilizer, and the like can be used depending on purposes. Examples of the additives include lactose, mannitol, crystalline cellulose, hydroxypropylcellulose having a low substitution degree, corn starch, partly pregelatinized starch, carmellose calcium, croscarmellose sodium, hydroxypropylcellulose, hydroxypropylmethylcellulose, polyvinyl alcohol, magnesium stearate, sodium stearyl fumarate, polyethylene glycol, propylene glycol, titanium oxide, and talc.

As the additives, a water-soluble additive is preferable. The use of the water-soluble additive may optimize the release rate of the semaphorin inhibitor and/or achieve the stabilization of the semaphorin inhibitor and the like. That is, the water-soluble additive enables the still more efficient deliver of the semaphorin inhibitor to the affected part.

In the present invention, it is preferable to add a water-soluble additive for further optimizing the release rate or for a purpose such as drug stabilization.

As long as the water-soluble additive is a solid at normal temperature, and is medically and pharmaceutically acceptable, the water-soluble additive is not particularly limited, and known water-soluble additives may be used. As long as the water-soluble additive is a solid at normal temperature, and is medically and pharmaceutically acceptable, the water-soluble additive is not limited, and one or more amino acids, sugars not having a primary amine, salts, and bile salts are preferable as the water-soluble additive in the sheet preparation of the present invention. Examples of these preferred water-soluble additives specifically include the following:
Examples of the one or more amino acids include neutral amino acids or hydrophobic amino acids. Among these amino acids, glycine, alanine, valine, leucine, and isoleucine are more preferable, which have an alkyl chain, and alanine and leucine are particularly preferable. Among alanine and leucine, L-alanine and L-leucine are preferable, respectively.
Examples of the sugars not having a primary amine include glucose, mannitol, lactose, trehalose, sucrose, erythritol, sorbitol, and xylitol, and preferably include glucose, mannitol, and lactose. Among these, mannitol is particularly preferable.
Examples of the salts include sodium chloride, potassium chloride, and calcium chloride, and preferably include sodium chloride.
Examples of the bile salts include sodium cholate and sodium chenodeoxycholate, which are primary bile salts, sodium desoxycholate and sodium lithocholate, which are secondary bile salts, and sodium glycocholate and sodium taurocholate, which are complex bile salts, and preferably include sodium cholate, sodium desoxycholate, and sodium glycocholate.

As the water-soluble additive used for the sheet preparation of the present invention, the one or more amino acids, sugars not having a primary amine, and salts are more preferable. Among these water-soluble additives, the one or more amino acids are particularly preferable. When sugars not having a primary amine or salts are used for the sheet preparation of the present invention, it is preferable to use these together.

Although the one or more amino acids used for the sheet preparation of the present invention are not limited, a neutral amino acid or a hydrophobic amino acid is preferable. Among these amino acids, glycine, alanine, valine, leucine, and isoleucine, which have an alkyl chain, are more preferable, and alanine and leucine are particularly preferable. Among sheet preparations of the present invention, a sheet preparation containing alanine or leucine as the one or more amino acids is preferable, and a sheet preparation of the present invention containing alanine and leucine is more preferable.

When the water-soluble additive is used for the sheet preparation of the present invention, its blending proportion is not limited, and is, for example, 5% by weight to 35% by weight, preferably 10% by weight to 25% by weight, and more preferably 15% by weight to 25% by weight.

When alanine is used as the water-soluble additive in the sheet preparation of the present invention, its blending proportion is not limited, and is, for example, 5% by weight to 25% by weight, preferably 8% by weight to 20% by weight, and more preferably 10% by weight to 20% by weight.

When leucine is used as the water-soluble additive in the sheet preparation of the present invention, its blending proportion is not limited, and is, for example, 0.5% by weight to 10% by weight, preferably 1% by weight to 10% by weight, and more preferably 1% by weight to 8% by weight.

When alanine and leucine are used as the water-soluble additive in the sheet preparation of the present invention, the blending ratio thereof is not limited, and the ratio of alanine:leucine is, for example, 10:1 to 2:1, preferably 8:1 to 2:1, and more preferably 8:1 to 3:1.

Amounts of Ingredients

The content of the semaphorin inhibitor in the sheet preparation of the present invention is not particularly limited, and may be 0.3 to 35%, and is preferably 2 to 20%, and more preferably 8 to 15%.

The content of the substrate in the sheet preparation of the present invention is not particularly limited, either, and is 30% to 90%, preferably 35% to 75%, and more preferably 40% to 60%.

When the water-soluble additive is used in the sheet preparation of the present invention, the content of the water-soluble additive to be added is not limited, and is 5% by weight to 35% by weight, and preferably 10% by weight to 25% by weight, and more preferably 15% by weight to 25% by weight.

Since the pliability is improved in the sheet preparation of the present invention further containing the one or more of amino acids among the sheet preparations of the present invention, hardening caused by the water-soluble drug such as the semaphorin inhibitor can be alleviated. Therefore, a larger amount of semaphorin inhibitor can be incorporated as an active ingredient into the sheet preparation of the present invention further containing the one or more amino acids among the sheet preparations of the present invention as compared with conventional sheet preparations.

Although the semaphorin inhibitor and the water-soluble additive suitably added are dispersed as powders in the carrier, the particle sizes thereof may affect releasability. Therefore, it is preferable to control the particle sizes of the semaphorin inhibitor and the water-soluble additive to a certain range as necessary to stabilize the quality of the sheet preparation of the present invention. The particle sizes as the upper limit are preferably 300 μm or less, and more preferably 200 μm or less. The particle sizes are preferably controlled to these particle sizes.

Size, Thickness, and Shape

The size and the shape of the sheet preparation of the present invention are not limited.

The size of the sheet preparation of the present invention is, for example, 2 to 90 mm in width and 2 to 140 mm in length, and the sheet preparation may be cut according to the size of an injured part at the time of use.

The thickness of the sheet preparation of the present invention is not limited, and may be, for example, 0.1 to 2.0 mm. The thickness of the sheet preparation of the present invention is preferably 0.3 to 1.5 mm, and more preferably 0.5 to 1.2 mm.

As long as the shape of the sheet preparation of the present invention is a shape which can be placed and fixed near the injured part, the shape is not limited, and examples of the whole shape include a round shape, an elliptical shape, and a rectangular shape.

The thickness of the sheet preparation can be measured with a slide calipers or the like after silicone hardening as a method for measuring the thickness of the sheet preparation of the present invention in experimental small-scale production, but silicone is elastic, and thus the thickness of the sheet preparation needs to be measured carefully so that shrinkage or deformation is not caused by excessive pressurization. Examples of a measuring method having little influence of pressurization include measurement under a microscope and an ultrasonic thickness meter. Although the thickness can also be measured at both points of immediately after formation before silicone hardening and after hardening in a production process, deformation is easily caused by pressurization before hardening, still more caution is therefore required. The sizes of metallic molds such as a nozzle, a slit, and a roller which are used for formation, and the rate of expansion at normal pressure are calculated beforehand, and the sheet preparation can also be produced with the size of a finished product estimated.

Production Method

A method for producing the sheet preparation of the present invention is not limited, and the sheet preparation may be produced by a method commonly used in the art. A method for producing a silicone preparation described, for example, in WO2012/018069 may be referred to for the sheet preparation in which silicone is used as a substrate among the sheet preparations of the present invention.

Use and Using Method

The sheet preparation of the present invention is used by applying the sheet preparation to the affected part of spinal cord injury or brain injury and/or its vicinity by epidural administration for treating spinal cord injury or brain injury. The term “treating” herein includes not only complete recovery but also being able to measure or recognize the alleviation of symptoms by objective indices and/or the subjectivity of patients.

Among the sheet preparations of the present invention, the sheet preparation having pliability, flexibility, and/or plasticity which is conformable to the curved shape of the spinal cord is particularly preferable in the treatment of spinal cord injury. To secure such pliability, flexibility, and/or plasticity, the sheet preparation of the present invention further containing the one or more amino acids is preferable.

The sheet preparation of the present invention may have a configuration further enhancing the fixability to the affected part using the sheet preparation by placing the substrate on a support layer. In the sheet preparation of the present invention, a side on which the substrate is placed among the two sides of the support layer may have an adhesive layer between the support layer and the substrate.

### EXAMPLES

Hereinafter, the present invention will be described in more detail by Examples with Preparation Examples and Test Examples. The present invention is not limited to these examples in any meaning.

### Preparation Example 1

Compound A, mannitol (PEARLITOL (Registered trademark) SD-Mannitol, manufactured by ROQUETTE), and sodium chloride (manufactured by NACALAI TESQUE, INC.) were weighed according to Table 1 and mixed uniformly in a mortar to obtain a mixed powder. Meanwhile, a Q7-4750 silicone A component (SILASTIC Q7-4750 silicone A component, manufactured by Dow Corning) and a Q7-4750 silicone B component (SILASTIC Q7-4750 silicone B component, manufactured by Dow Corning) were weighed according to Table 1 and kneaded with a two-roll mill. The above-mentioned silicone was kneaded, the whole amount of the above-mentioned mixed powder was then immediately added, and the mixture was kneaded. The mixture was then extended with the two-roll mill and hardened at 40° C. for 25 hours to obtain a sheet preparation having a thickness of 0.3 mm (Preparation Example 1). The “blending ratio (%)” indicates % by weight.

TABLE 1

| | Formulation | Blending ratio |
|---|---|---|
| Ingredient | (mg) | (%) |
| Compound A | 50 | 10 |
| Mannitol | 85 | 17 |
| Sodium chloride | 15 | 3 |
| Q7-4750 silicone A component | 175 | 35 |
| Q7-4750 silicone B component | 175 | 35 |
| Total | 500 | 100 |

### Preparation Example 2

According to Table 2, L-alanine (manufactured by NACALAI TESQUE, INC.), L-leucine (manufactured by NACALAI TESQUE, INC.), and compound A were weighed in this order, and a 12-mL ointment jar made of polypropylene was charged therewith. The powders were uniformly mixed using a microspatula to obtain a mixed powder. Meanwhile, 6.0 g of an MED-6215 silicone A component (manufactured by NuSil) and 0.6 g of an MED-6215 silicone B component (manufactured by NuSil) were weighed in a 10-mL syringe made of polypropylene, and the syringe was then attached to one side of a syringe mixer made of stainless steel. An empty 10-mL syringe made of polypropylene was attached to the other side, and the syringes were then fully deaerated. The syringes were manually pumped through the syringe mixer by making the syringes make 15 round-trips (30 times) for mixing to prepare a silicone mixture. Then, 0.936 g of this silicone mixture (0.851 gas the A component and 0.085 g as the B component) was weighed, and the above-mentioned ointment jar was charged therewith. The ointment jar was set in a rotating and revolving mixer (ARE-310, manufactured by THINKY CORPORATION), and the mixture was kneaded in a kneading mode at 2000 rpm for 2 minutes, in a centrifugal mode at 2000 rpm for 1 minute, and in the kneading mode at 2000 rpm for 2 minutes sequentially. The kneaded material was fully kneaded again using the microspatula, the whole amount of the kneaded material was collected in a 5-mL syringe made of polypropylene, the syringe was then set in a centrifuge (CF7D2, manufactured by Koki Holdings Co., Ltd.), and the kneaded material was defoamed under the conditions of 1000 rpm and 2 minutes. The defoamed kneaded material was injected into a SUS mold having a thickness of 1.05 mm, the mold was installed in a manual hydraulic hot press (manufactured by Imoto machinery Co., LTD), and the defoamed kneaded material was hardened under conditions of 100° C. and 30 minutes with a load of 0.8 ton (9.8 MPa) applied to obtain a sheet preparation having a thickness of 1 mm (Preparation Example 2).

TABLE 2

| | Formulation | Blending ratio |
|---|---|---|
| Ingredient | (g) | (%) |
| Compound A | 0.54 | 30 |
| L-Alanine | 0.27 | 15 |
| L-Leucine | 0.054 | 3 |
| MED-6215 silicone A component | 0.851 | 47.3 |
| MED-6215 silicone B component | 0.085 | 4.7 |
| Total | 1.8 | 100 |

### Preparation Example 3

According to Table 3, L-alanine (manufactured by NACALAI TESQUE, INC.), and L-leucine (manufactured by NACALAI TESQUE, INC.) were weighed in this order, and a 12-mL ointment jar made of polypropylene was charged therewith. The powders were uniformly mixed using a microspatula to obtain a mixed powder. Meanwhile, 6.0 g of an MED-6215 silicone A component (manufactured by NuSil) and 0.6 g of an MED-6215 silicone B component (manufactured by NuSil) were weighed in a 10-mL syringe made of polypropylene, the syringe was then attached to one side of a syringe mixer made of stainless steel. An empty 10-mL syringe made of polypropylene was attached to the other side, and the syringes were then fully deaerated. The syringes were manually pumped through the syringe mixer by making the syringes make 15 round-trips (30 times) for mixing to prepare a silicone mixture. Then, 1.476 g of this silicone mixture (1.342 gas the A component and 0.134 g as the B component) was weighed, and the above-mentioned ointment jar was charged therewith. The ointment jar was set in a rotating and revolving mixer (ARE-310, manufactured by THINKY CORPORATION), and the mixture was kneaded in a kneading mode at 2000 rpm for 2 minutes, in a centrifugal mode at 2000 rpm for 1 minute, and in the kneading mode at 2000 rpm for 2 minutes sequentially. The kneaded material was fully kneaded again using the microspatula, the whole amount of the kneaded material was collected in a 5-mL syringe made of polypropylene, the syringe was then set in a centrifuge (CF7D2, manufactured by Koki Holdings Co., Ltd.), and the kneaded material was defoamed under the conditions of 1000 rpm and 2 minutes. The defoamed kneaded material was injected into a SUS mold having a thickness of 1.05 mm, the mold was installed in a manual hydraulic hot press (manufactured by Imoto machinery Co., LTD), and the defoamed kneaded material was hardened under conditions of 100° C. and 30 minutes with a load of 0.8 ton (9.8 MPa) applied to obtain a sheet preparation having a thickness of 1 mm (Preparation Example 3).

TABLE 3

| | Formulation | Blending ratio |
|---|---|---|
| Ingredient | (g) | (%) |
| Compound A | — | — |
| L-Alanine | 0.27 | 15 |
| L-Leucine | 0.054 | 3 |
| MED-6215 silicone A component | 1.342 | 74.5 |
| MED-6215 silicone B component | 0.134 | 7.5 |
| Total | 1.8 | 100 |

### [Test Example 1] Drug Release Test on Preparation Example 1

The sheet of Preparation Example 1 was cut in a rectangle of 5 mm×7 mm, and the cut sheet was used as Test Preparation 1-1. The cut Test Preparation 1-1 was put into 1 mL of phosphate buffered saline (PBS) and left to stand at 25° C., compound A released from the preparation was quantified by ultra fast liquid chromatography (UFLC, manufactured by SHIMADZU CORPORATION), and the cumulative release rate was calculated.

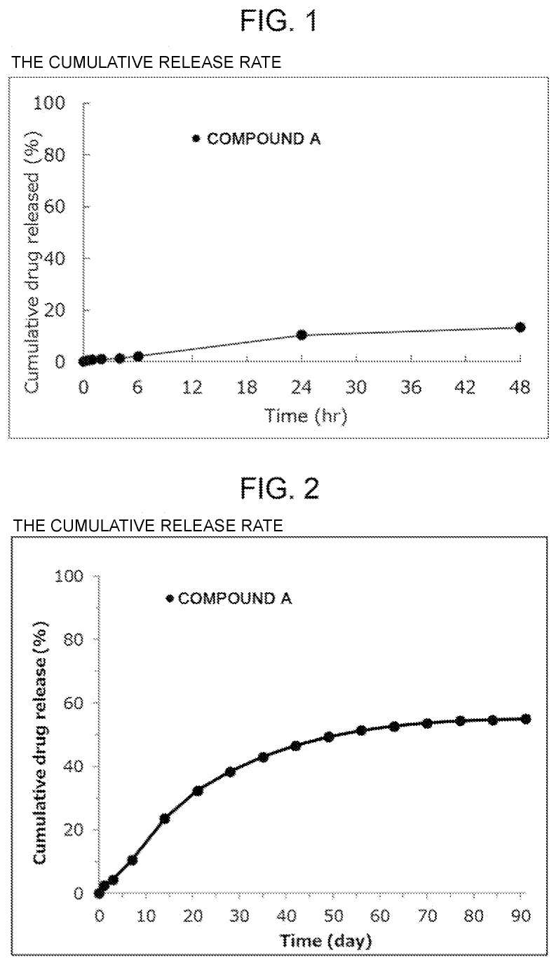

Consequently, drug release as shown in FIG. 1 was shown.

### [Test Example 2] Drug Release Test on Preparation Example 2

The sheet of Preparation Example 2 was cut in a square of 3 mm×3 mm, and the cut sheet was used as Test Preparation 2-1. The sheet was tested by the same method as in Test Example 1, and the cumulative release rate of compound A from the preparation was calculated.

Consequently, as shown in FIG. 2, the good sustained release which reaches 90 days was achieved.

### [Example 1] Hind-Limb Motor Function Evaluation (BBB Score Evaluation) Test Using Rat Spinal Cord Injury Model

A hind-limb motor function evaluation (BBB score evaluation, refer to Basso D M, Beattie M S, Bresnahan J C. A sensitive and reliable locomotor rating scale for open field testing in rats. J. Neurotrauma 1995; 12:1-21.) test using 7-week old female SD rat spinal cord injury model was performed. The skin on the back of the rat was opened under deep anesthetization, the spinous process and the vertebral arch of the tenth thoracic vertebra were excised to a diameter of around 2.5 mm, and the exposed spinal cord was subjected to crush injury using an IH impactor (Brain Science idea. Co., Ltd.) at a force of 250 kdyn. Then, it was confirmed that there was no injury on the dura mater macroscopically, and tissue surrounding the spinal cord did not bleed abnormally to produce a spinal cord injury model. The sheet of Preparation Example 2 was cut in a square of 3 mm×3 mm, and the cut sheet was used as Test Preparation 2-2. As Comparative Example, the sheet of Preparation Example 3 was cut in a square of 3 mm×3 mm, and the cut sheet was used as Test Preparation 3-2. The cut Test Preparation was administered (indwelled) to the dura mater on the injured spinal cord immediately after spinal cord injury. Rehabilitation treatment was performed using a treadmill from the seventh day after the spinal cord injury at a frequency of 20 minutes/day and 5 days/week. The rat was made to wear a jacket which can support the front body by passing the fore-limbs through the jacket and supported at a height at which the fore-limbs and the hind-limbs could be placed lightly on the treadmill (Natsume Seisakusho Co., Ltd., KN-73). The belt of the treadmill was moved at a speed of 0.6 m/min from the seventh day after the spinal cord injury, the hind-limbs of the rat were compulsorily moved for 20 minutes, and this exercise was considered as the rehabilitation for hind-limb motor function recovery. The belt speed was increased to 1.8 m/min 2 weeks thereafter and to 3.0 m/min further 2 weeks thereafter, and the rehabilitation was continued. BBB score evaluation was performed weekly from 1 week after the spinal cord injury to the thirteenth week. The BBB score evaluation was performed in accordance with the method of Basso et al 1). In the second week after spinal cord injury, animals having BBB scores of 9 or more were considered as spontaneously restored animals and excluded from the test.

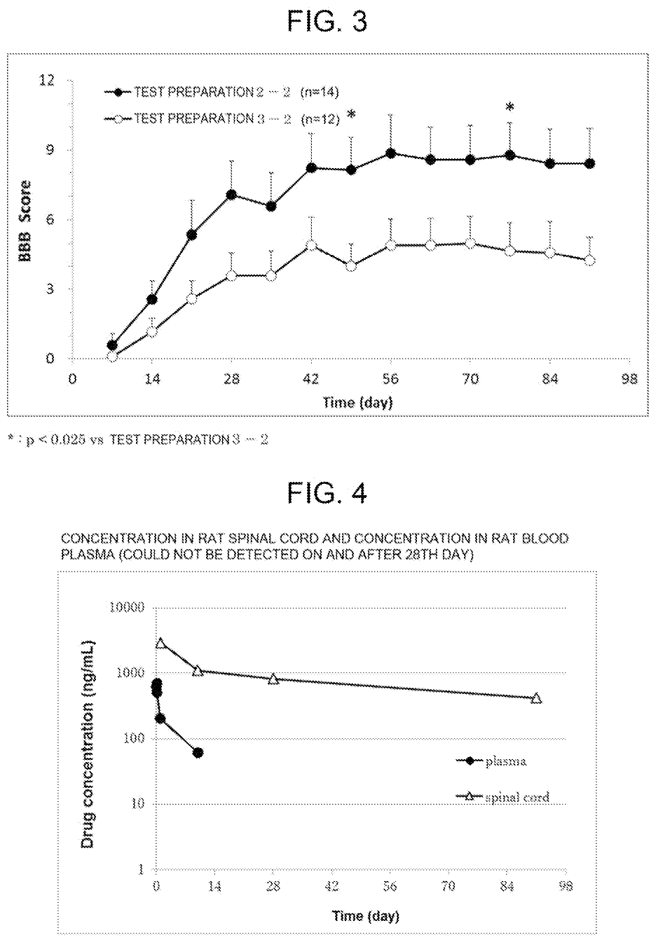

As shown in FIG. 3, compound A administration group (Test Preparation 2-2) which is Example of the present invention consequently exhibited significant improvement in a BBB score in the seventh and eleventh weeks as compared with Comparative Example (Test Preparation 3-2).

### [Example 2] Evaluation Test on Drug Transfer to Spinal Cord by Indwelling Outside Rat Dura Mater Spinalis

Drug transfer from a sheet preparation indwelled outside the dura mater spinalis to spinal cord tissue was evaluated using 7-week old female SD rats. The sheet of Preparation Example 2 was cut in a square of 3 mm×3 mm, and the cut sheet was used as Test Preparation 2-3. The skin on the back of each rat was opened under deep anesthetization, the spinous process and the vertebral arch of the tenth thoracic vertebra were excised, and the dura mater spinalis was exposed. Test Preparation 2-3 was immediately administered (indwelled) to the dura mater spinalis, and the opened part was closed. The spinal cords were collected 1, 10, 28, and 91 days after the indwelling, and the concentrations of compound A in the tissue were measured by LC/MS/MS (API-4000, AB Sciex Pte. Ltd.). Furthermore, blood was collected 1, 3, 6, and 24 hours, and 10, 28, and 91 days after the indwelling, and the concentrations of compound A in blood plasma were quantified by the above-mentioned LC/MS/MS.

As shown in FIG. 4, it was consequently shown that an adequate amount of the drug administered epidurally was transferred to spinal cord tissue. Meanwhile, it was suggested that the concentrations in blood plasma were lower as compared with the concentrations in the spinal cord, and the drug was directly dispersed in the spinal cord without being transferred to blood.

### [Example 3] Evaluation Test on Canine and Swine Cerebral Dura Mater Permeability Using Franz Cell

A canine (beagle) and a swine (Gottingen mini-pig) were exsanguinated and slaughtered under deep anesthetization, the cerebral dura maters were then collected. The receptor chamber side of a franz cell (manufactured by Keystone Scientific K. K.) was fixed on 6 continuous stirrers, the receptor chamber was connected with a thermostatic bath by a tube, and warm water at 37° C. was made to flow back in a water jacket. The receptor chamber was charged with a rotor, and the chamber was then filled with artificial cerebrospinal fluid (ACSF). The temperature of ACSF in the chamber stabilized, each dura mater was then placed on the receptor chamber, and a donor chamber was further set from above the dura mater and fixed with clamps. The amount of the ACSF in the receptor chamber was adjusted to 5 mL in accordance with a marked line. Preparation Example 1 was cut in a rectangle of 5 mm×7 mm, and the cut Preparation Example 1 was used as Test Preparation 1-2. The cut Test Preparation 1-2 was indwelled on the dura mater. Furthermore, the top of the donor chamber was sealed with parafilm for dry prevention, and the test was started. Then, 300 μL was sampled from a sampling port over time, and an equivalent amount of ACSF was filled from the sampling port immediately after sampling. The sampling was performed 7 times at the time points of 0.5, 1, 2, 4, 6, 24, and 48 hours. The concentrations of compound A in sampled solutions were quantified by ultra fast liquid chromatography (UFLC, manufactured by SHIMADZU CORPORATION), and the amounts transferred were calculated.

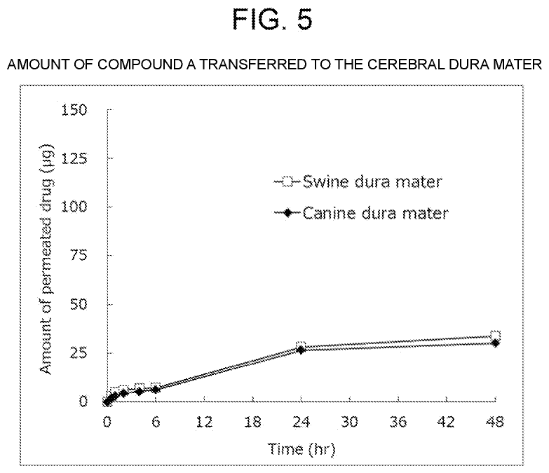

As shown in FIG. 5, it was shown that compound A was released from Test Preparation 1-2, which was Example of the present invention obtained from Preparation Example 1, and was transferred to the cerebral dura mater regardless of the difference in the animal species.

Methods for synthesizing compound A will be illustrated below.

### Synthesis Example 1

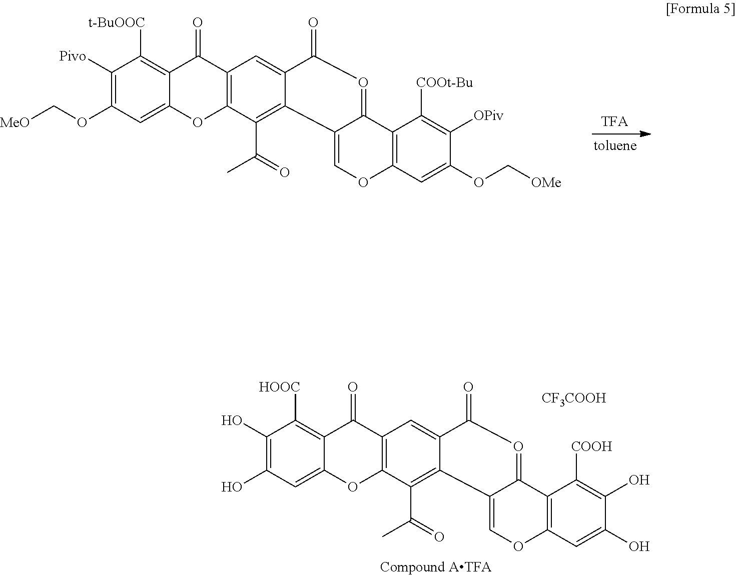

A protected compound A (160 g, 169 mmol) synthesized by the method described in a literature (ACS Chemical Neuroscience 2015, 6, 542-550), toluene (800 g), and water (6.10 g, 0.339 mmol) were added to a reaction container, and the mixture was kept at 25° C. Trifluoroacetic acid (1.54 kg, 13.5 mol) was dropped thereon, and seed crystals (1.60 g, 1.69 mmol) were added. The mixture was stirred at 40° C. for 1 hour, the temperature was raised to 60° C., and the mixture was stirred for further 3 hours. The mixture was cooled to 0° C., ethanol (400 g) was added, the mixture was stirred all night, and the produced crystals were filtered off and collected. The residue was washed with toluene (320 g) and ethanol (320 g×twice) sequentially and suction-dried at 40° C. to obtain compound A trifluoroacetic acid solvate (107.9 g, yield 92.3% as a mono-TFA solvate) as a yellow solid.

XRD: 2θ=7.5, 8.4, 10.0, 12.0, 14.0, 14.2, 14.9, 21.8, 22.4, 23.9.

TGA: The weight decreased by 15.0% from 30° C. to 190° C.

### Synthesis Example 2

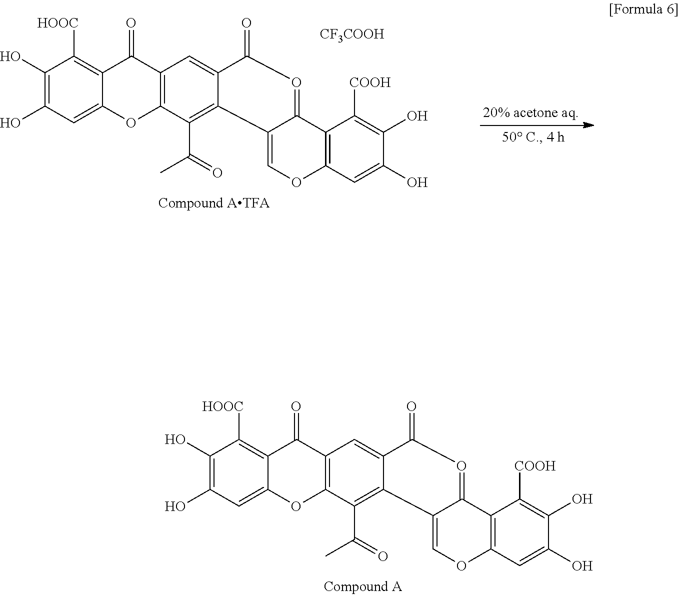

Compound A trifluoroacetic acid solvate (105 g, 152 mmol) obtained in Synthesis Example 1, acetone (210 g), and deionized water (840 g) were mixed, and the mixture was stirred at 50° C. for 4 hours. The mixture was cooled to 0° C. over 2 hours and stirred for further 1 hour. The produced crystal was filtered off and collected, washed with a mixed solvent of acetone/water (31.5 g/126 g) twice, washed with a small amount of acetone once and suction-dried at 40° C. to obtain compound A (92.2 g, quantitative, 2-step yield 97.2%) as a yellow solid.

A nuclear magnetic resonance (NMR) spectrum was measured using an AV400M (400 MHz) manufactured by Bruker BioSpin. X-ray powder diffraction (XRD) was measured using a D8 ADVANCE manufactured by Bruker AXS in a diffraction angle range of 20=5° to 40° under conditions of a Cu Kα line, an X-ray tube current of 40 mA, a voltage of 40 kV, a step of 0.015°, and a measuring time of 48 seconds/step. TGA (thermogravimetric analysis) was measured using a Q500 manufactured by TA Instruments Japan Inc. in a platinum pan container under such conditions that the measurement temperature ranged from room temperature to 300° C., the rate of temperature increase was 10° C./minute, the atmosphere gas was dry nitrogen, the sample flow rate was around 60 mL/minute, and the balance flow rate was around 40 mL/minute.

¹H-NMR (DMSO-d₆, 400 MHz) δ: 11.65 (1H, brs), 11.40 (1H, brs), 9.41 (2H, brs), 8.53 (1H, s), 8.17 (1H, s), 6.96 (1H, s), 6.94 (1H, s), 2.54 (1H, s), 2.53 (1H, s).

XRD: 2θ=7.57, 8.22, 13.2, 13.7, 21.5, 22.2, 22.825, 0, 27.9, 30.0.

### Synthesis Example 3

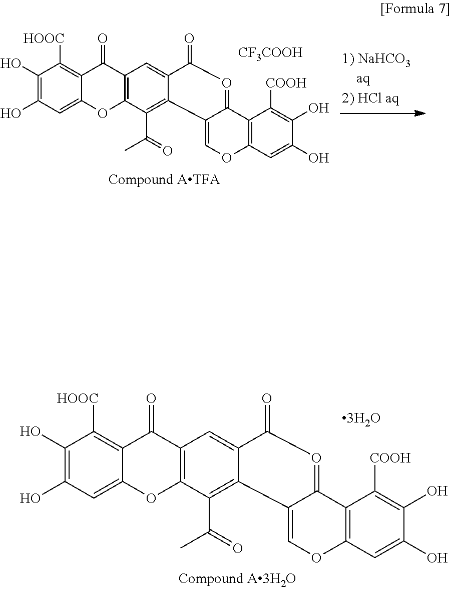

An aqueous 3% sodium hydrogen carbonate solution (52.7 g, 18.7 mmol) was added to slurry obtained by mixing 5% acetone water (5.00 g) with compound A trifluoroacetic acid solvate (1.00 g, 1.44 mmol) obtained in Synthesis Example 1, and the mixture was stirred at 25° C. for 1 hour for dissolution. This solution was dropped into 9% hydrochloric acid (8.21 g, 20.1 mmol) over 30 minutes. The mixture was stirred at 25° C. for 3 hours, the produced crystals were filtered off and collected, washed with water (2.00 g) 3 times, and then suction-dried at 25° C. for 3 hours. The crystals were left to stand in a container containing a saturated aqueous potassium chloride solution (at a humidity of 80% or more), and the humidity was controlled all night to obtain compound A trihydrate (0.900 g, yield 98.5%) as a pale yellow solid.

XRD: 2θ=10.1, 15.5, 19.1, 24.0, 25.6.

TGA: The weight decreased by 8.35% from 25° C. to 125° C.

### Synthesis Example 4

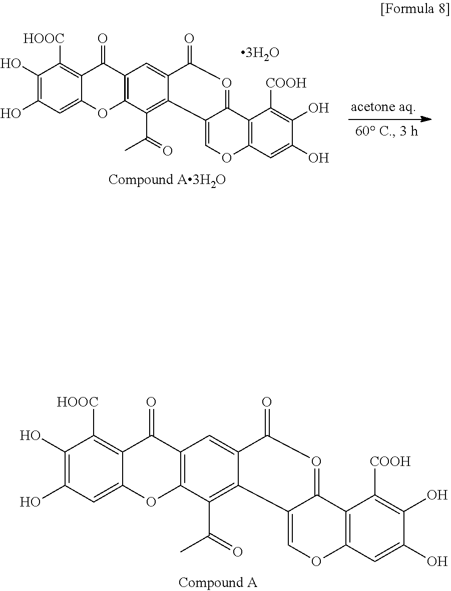

Compound A trihydrate (1.25 g, 1.98 mmol) obtained in Synthesis Example 3 was mixed with 50% by weight acetone water (37.5 g), and the mixture was heated and stirred at 60° C. for 3 hours. The mixture was cooled to 0° C. over 1 hour and stirred for further 4 hours. The crystals were filtered off and collected, washed with 50% by weight acetone water (1.88 g) twice, and then suction-dried at 40° C. to obtain compound A (1.01 g, yield 88.6%) as a yellow solid.

### INDUSTRIAL APPLICABILITY

According to the present invention, the application to an affected part of spinal cord injury or brain injury and/or its vicinity enables a semaphorin inhibitor to reach the affected part without removing the dura mater surgically. That is, the use of a sheet preparation of the present invention enables the treatment of spinal cord injury or brain injury with a physical burden of a patient greatly reduced.

1. A sheet preparation for treating spinal cord injury or brain injury by epidural administration, comprising a semaphorin inhibitor.

2. The sheet preparation according to claim 1, comprising silicone as a substrate.

3. The sheet preparation according to claim 1, further comprising a water-soluble additive.

4. The sheet preparation according to claim 3, comprising one or more amino acids as the water-soluble additive.

5. The sheet preparation according to claim 4, wherein the one or more amino acids are alanine or leucine.

6. The sheet preparation according to claim 1, wherein the semaphorin inhibitor is a semaphorin 3A inhibitor.

7. The sheet preparation according to claim 1, wherein the semaphorin inhibitor is compound A represented by formula (1):

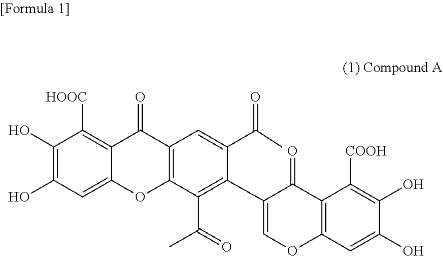 [SMILES: CC(=O)c1cc2c(=O)c3c(C(=O)O)c(O)c(O)cc3oc2c(C(C)=O)c1-c1coc2cc(O)c(O)c(C(=O)O)c2c1=O]

8. A sheet preparation, comprising compound A represented by formula (1):

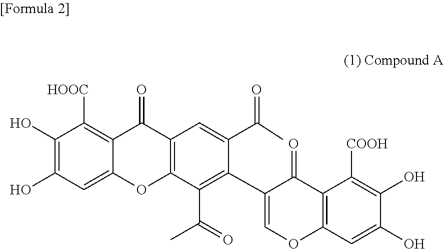 [SMILES: CC(=O)c1cc2c(=O)c3c(C(=O)O)c(O)c(O)cc3oc2c(C(C)=O)c1-c1coc2cc(O)c(O)c(C(=O)O)c2c1=O]

9. The sheet preparation according to claim 8, comprising silicone as a substrate.

10. The sheet preparation according to claim 8, further comprising a water-soluble additive.

11. The sheet preparation according to claim 10, comprising one or more amino acids as the water-soluble additive.

12. The sheet preparation according to claim 11, wherein the one or more amino acids are alanine or leucine.

13. A method for treating spinal cord injury or brain injury, wherein a therapeutically effective amount of the sheet preparation according to claim 8 is epidurally administered to a patient in need of treatment.

14. Use of the sheet preparation according to claim 8 for producing a therapeutic agent for spinal cord injury or brain injury to be epidurally administered.

15. A therapeutic agent for spinal cord injury or brain injury, comprising the sheet preparation according to claim 8, wherein the therapeutic agent is epidurally administered.
